(19) United States
(12) Patent Application Publication
(10) Pub. No.: US 2003/0032807 A1
(43) Pub. Date: Feb. 13, 2003

(54) Method for the production of 1-amino -3-aryl -uracils

(76) Inventors: Roland Andree, Langenfeld (DE); Dorothee Hoischen, Dusseldorf (DE); Achim Hupperts, Dusseldorf (DE); Karl-Heinz Linker, Leverkusen (DE); Holger Weintritt, Langenfeld (DE); Heinz-Jurgen Wroblowsky, Langenfeld (DE)

Correspondence Address: BAYER CORPORATION, PATENT DEPARTMENT, 100 BAYER ROAD, PITTSBURGH, PA, 15205, US

(21) Appl. No.: 10/182,966

(22) PCT Filed: Jan. 25, 2001
(86) PCT No.: PCT/EP01/00795

(30) Foreign Application Priority Data
Feb. 7, 2000 (DE) 10005284.3

(51) Int. Cl. C07D 239/52

(52) U.S. Cl. 544/316

(57) ABSTRACT

The invention relates to a novel process for preparing 1-amino-3-aryluracils of the formula (I)

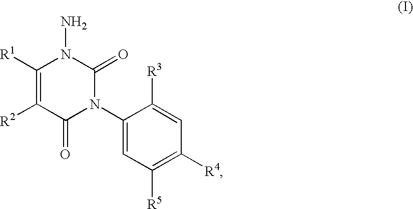

in which

R¹ is optionally substituted alkyl,

R² is hydrogen, nitro, cyano, halogen or optionally substituted alkyl,

R³ is hydrogen, nitro, cyano or halogen,

R⁴ is hydrogen, nitro, cyano, carbamoyl, thiocarbamoyl, hydroxyl or halogen, or optionally substituted alkyl, alkoxy or benzoyloxy,

R⁵ is hydrogen, hydroxyl, mercapto, amino, hydroxyamino, nitro, cyano, carboxyl, carbamoyl, thiocarbamoyl or halogen, or is one of the following moieties

—R⁶, —Q—R⁶, —NH—R⁶, —NH—O—R⁶, —NH—SO₂—R⁶, —N(SO₂R⁶)₂, —CQ¹—R⁷, —CQ¹—Q²—R⁶, —CQ¹—NH—R⁶, —Q²—CQ¹—R⁶, —Q²—CQ¹—Q²—R⁶, —NH—CQ¹—R, —N(SO₂—R⁶)—(CQ¹—R⁶), —NH—CQ¹—Q²—R⁶, —Q²—CQ¹—NH—R⁶

where

Q is O, S, SO or SO₂, p2 Q¹ and Q² are independently O or S, and

R⁶ is alkyl, alkenyl, alkinyl, cycloalkyl, cycloalkylalkyl, aryl, arylalkyl, heterocyclyl or heterocyclylalkyl, each of which is optionally substituted.

The invention relates to a novel process for preparing 1-amino-3-aryluracils which are well known as active ingredients in agrochemicals or as intermediates for preparing active ingredients.

It is well known that 1-amino-3-aryluracils are obtained when 3-aryluracils are reacted with 1-aminooxy-2,4-dinitrobenzene (cf. WO-A-94/04511, WO-A-95/29168, WO-A-96/36614, WO-A-97/05116, WO-A-98/06706, WO-A-98/25909). However, amination using 1-aminooxy-2,4-dinitrobenzene requires large excesses of this aminating agent and in many cases delivers only unsatisfactory yields after long reaction times.

It is also well known that 2-aminooxysulphonyl-1,3,5-trimethylbenzene (O-mesitylenesulphonylhydroxylamine) can be used instead of 1-aminooxy-2,4-dinitrobenzene for N-amination (cf. WO-A-97/08170, U.S. Pat. No. 5,661,108). However, the yield and quality of the products obtained in this way are not entirely satisfactory.

It was found that 1-amino-3-aryluracils of the formula (I)

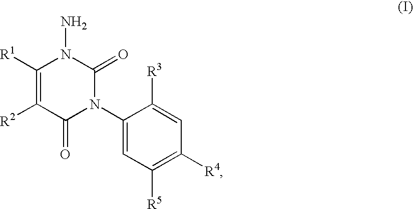

in which

R¹ is optionally substituted alkyl,

R² is hydrogen, nitro, cyano, halogen or optionally substituted alkyl,

R³ is hydrogen, nitro, cyano or halogen,

R⁴ is hydrogen, nitro, cyano, carbamoyl, thiocarbamoyl, hydroxyl or halogen, or optionally substituted alkyl, alkoxy or benzoyloxy,

R⁵ is hydrogen, hydroxyl, mercapto, amino, hydroxyamino, nitro, cyano, carboxyl, carbamoyl, thiocarbamoyl or halogen, or is one of the following moieties

—R⁶, —Q—R⁶, —NH—R⁶, —NH—O—R⁶, —NH—SO₂—R⁶, —N(SO₂R⁶)₂, —CQ¹—R⁷, —CQ¹—Q²—R⁶, —CQ¹—NH—R⁶, —Q²—CQ¹—R⁶, —Q²—CQ¹—Q²—R⁶, —NH—CQ¹—R⁶, —N(SO₂—R⁶)—(CQ¹—R⁶), —NH—CQ¹—Q²—R⁶, —Q²—CQ¹—NH—R⁶

where

Q is O, S, SO or SO₂,

Q¹ and Q² are independently O or S, and

R⁶ is alkyl, alkenyl, alkinyl, cycloalkyl, cycloalkylalkyl, aryl, arylalkyl, heterocyclyl or heterocyclylalkyl, each of which is optionally substituted, are obtained in good yields and good quality when 3-aryluracils of the formula (II)

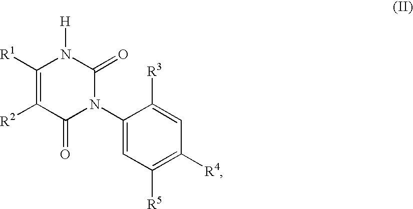

in which

R¹, R², R³, R⁴ and R⁵ have the above meanings,

are reacted with 2-aminooxysulphonyl-1,3,5-trimethylbenzene (O-mesitylene-sulphonylhydroxylamine) of the formula (III)

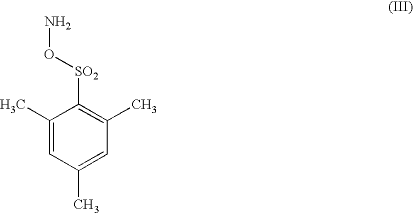

optionally in the presence of a reaction auxiliary and optionally in the presence of a diluent at temperatures in the range from −50° C. to 80° C.

Surprisingly, the 1-amino-3-aryluracils of the general formula (I) are obtained by the process of the invention in substantially better yields (in comparison with the known processes employing 1-aminooxy-2,4-dinitrobenzene) after considerably shortened reaction times.

Since the space-time yields of the process of the invention are greatly improved in comparison with the prior art methodology, it is a substantial advance in the art.

Preferred meanings for the moieties, radicals or substituents described above and below are as follows:

R¹ is preferably optionally halogen-substituted alkyl having from 1 to 4 carbon atoms,

R² is preferably hydrogen, nitro, cyano, halogen or optionally halogen-substituted alkyl having from 1 to 4 carbon atoms,

R³ is preferably hydrogen, nitro, cyano, fluorine, chlorine or bromine,

R⁴ is preferably hydrogen, nitro, cyano, carbamoyl, thiocarbamoyl, hydroxyl or halogen, or is alkyl or alkoxy having from 1 to 4 carbon atoms, each of which is optionally substituted by halogen, or is optionally halogen-, C₁-C₄-alkyl- or C₁-C₄-alkoxy-substituted benzoyloxy and

R⁵ is preferably hydrogen, hydroxyl, mercapto, amino, hydroxyamino, nitro, cyano, carboxyl, carbamoyl, thiocarbamoyl, halogen, or is one of the following moieties —R⁶, —Q—R⁶, —NH—R⁶, —NH—O—R⁶, —NH—SO₂—R⁶, —N(SO₂R⁶)₂, —CQ¹—R⁷, —CQ¹—Q²—R⁶, —CQ¹—NH—R⁶, Q²CQ¹—R⁶, —Q²—CQ¹—Q²R⁶, —NH—CQ¹—R⁶, —N(SO₂—R⁶)(CQ¹—R⁶), —NH—CQ¹Q²—R⁶, —Q²—CQ¹—NH—R⁶,

where preferably

Q is O, S, SO or SO₂,

Q¹ and Q² are independently O or S, and

R⁶ is alkyl having from 1 to 6 carbon atoms, which is optionally substituted by cyano, halogen, C₁-C₄-alkoxy, C₁-C₄-alkylthio, C₁-C₄-alkylcarbonyl, C₁-C₄-alkoxycarbonyl or C₁-C₄-alkylaminocarbonyl,

or is alkenyl or alkinyl having from 2 to 6 carbon atoms, each of which is optionally substituted by cyano, carboxyl, halogen, C₁-C₄-alkylcarbonyl, C₁-C₄-alkoxycarbonyl or C₁-C₄-alkylaminocarbonyl,

or is cycloalkyl or cycloalkylalkyl having from 3 to 6 carbon atoms in the cycloalkyl group and optionally from 1 to 4 carbon atoms in the alkyl part, each of which is optionally substituted by cyano, carboxyl, halogen, C₁-C₄-alkylcarbonyl or C₁-C₄-alkoxycarbonyl,

or is aryl or arylalkyl having 6 or 10 carbon atoms in the aryl group and optionally from 1 to 4 carbon atoms in the alkyl part, each of which is optionally substituted by from one to three substituents selected from the group consisting of hydroxyl, mercapto, amino, cyano, carboxyl, carbamoyl, thiocarbamoyl, C₁-C₄-alkyl, C₁-C₄-halogenalkyl, C₁-C₄-alkoxy, C₁-C₄-halogenalkoxy, C₁-C₄-alkylthio, C₁-C₄-halogenalkylthio, C₁-C₄-alkylsulphinyl, C₁-C₄-alkylsulphonyl, C₁-C₄-alkylamino and dimethylamino,

or is heterocyclyl or heterocyclylalkyl having from 2 to 6 carbon atoms and from 1 to 3 nitrogen atoms and/or 1 or 2 oxygen atoms and/or a sulphur atom in the heterocyclyl group and optionally from 1 to 4 carbon atoms in the alkyl part, each of which is optionally substituted by from one to three substituents selected from the group consisting of hydroxyl, mercapto, amino, cyano, carboxyl, carbamoyl, thiocarbamoyl, C₁-C₄-alkyl, C₁-C₄-halogenalkyl, C₁-C₄-alkoxy, C₁-C₄-halogenalkoxy, C₁-C₄-alkylthio, C₁-C₄-halogenalkyl-thio, C₁-C₄-alkylsuphinyl, C₁-C₄-alkylsulphonyl, C₁-C₄-alkylamino and dimethylamino.

R¹ is more preferably methyl, ethyl, n- or i-propyl, each of which is optionally substituted by fluorine and/or chlorine,

R² is more preferably hydrogen, nitro, cyano, fluorine, chlorine or bromine, or is methyl or ethyl, each of which is optionally substituted by fluorine and/or chlorine,

R³ is more preferably hydrogen, fluorine or chlorine,

R⁴ is more preferably hydrogen, nitro, cyano, carbamoyl, thiocarbamoyl, hydroxyl, fluorine, chlorine or bromine, or is methyl or methoxy, each of which is optionally substituted by fluorine and/or chlorine, and

R⁵ is more preferably hydrogen, hydroxyl, mercapto, amino, hydroxyamino, nitro, cyano, carboxyl, carbamoyl, thiocarbamoyl, fluorine, chlorine, bromine, iodine, or one of the following moieties —R⁶, —Q—R⁶, —NH—R⁶, —NH—O—R⁶, —NH—SO₂—R⁶, —N(SO₂R⁶)₂, —CQ¹—R⁷, —CQ¹—Q²—R⁶, —CQ¹—NH—R⁶, Q²—CQ¹—R⁶, —Q²—CQ¹Q²—R⁶, —NH—CQ¹—R⁶, —N(SO₂—R⁶)(CQ¹—R⁶), —NH—CQ¹—Q²R⁶, —Q²CQ¹—NH—R⁶,

where more preferably

Q is O, S, SO or SO₂,

Q¹ and Q² are independently O or S, and

R⁶ is methyl, ethyl, n- or i-propyl, n-, i-, s- or t-butyl, each of which is optionally substituted by cyano, fluorine, chlorine, methoxy, ethoxy, methylthio, ethylthio, acetyl, propionyl, methoxycarbonyl, ethoxycarbonyl, methylamino-carbonyl or ethylaminocarbonyl,

or is propenyl, butenyl, propinyl or butinyl, each of which is optionally substituted by cyano, carboxyl, fluorine, chlorine, bromine, acetyl, propionyl, n- or i-butyroyl, methoxycarbonyl, ethoxycarbonyl, n- or i-propoxycarbonyl, methylaminocarbonyl, ethylaminocarbonyl or n- or i-propylaminocarbonyl,

or is cyclopropyl, cyclobutyl, cyclopentyl, cyclohexyl, cyclopropylmethyl, cyclobutylmethyl, cyclopentylmethyl or cyclohexylmethyl, each of which is optionally substituted by cyano, carboxyl, fluorine, chlorine, bromine, acetyl, propionyl, methoxycarbonyl or ethoxycarbonyl,

or is phenyl, benzyl or phenylethyl, each of which is optionally substituted by from one to three substituents selected from the group consisting of hydroxyl, mercapto, amino, cyano, carboxyl, carbamoyl, thiocarbamoyl, methyl, ethyl, trifluoromethyl, methoxy, ethoxy, difluoromethoxy, trifluoromethoxy, methylthio, ethylthio, difluoromethylthio, trifluoromethylthio, methylsulphinyl, ethylsulphinyl, methylsulphonyl, methylamino, ethylamino and dimethylamino,

or is heterocyclyl or heterocyclylalkyl selected from the group consisting of oxiranyl, oxetanyl, furyl, tetrahydrofuryl, dioxolanyl, thienyl, tetrahydrothienyl, pyrrolyl, pyrazolyl, imidazolyl, triazolyl, oxazolyl, isoxazolyl, thiazolyl, isothiazolyl, oxadiazolyl, thiadiazolyl, pyridinyl, pyrimidinyl, triazinyl, pyrazolylmethyl, furylmethyl, thienylmethyl, oxazolylmethyl, isoxazolylmethyl, thiazolylmethyl, pyridinylmethyl and pyrimidinylmethyl, each of which is optionally substituted by one or two substituents selected from the group consisting of hydroxyl, mercapto, amino, cyano, carboxyl, carbamoyl, thiocarbamoyl, methyl, ethyl, n- or i-propyl, n-, i-, s- or t-butyl, difluoromethyl, dichloromethyl, trifluoromethyl, trichloromethyl, chlorodifluoromethyl, fluorodichloromethyl, methoxy, ethoxy, difluoro-methoxy, trifluoromethoxy, methylthio, ethylthio, difluoromethylthio, trifluoromethylthio, methylsulphinyl, ethylsulphinyl, methylsulphonyl, ethylsulphonyl, methylamino, ethylamino and dimethylamino.

R¹ is particularly preferably trifluoromethyl.

R² is particularly preferably hydrogen, chlorine or methyl.

R³ is particularly preferably fluorine or chlorine.

R⁴ is particularly preferably cyano, carbamoyl, thiocarbamoyl, hydroxyl, fluorine, chlorine, bromine or trifluoromethyl.

R⁵ is particularly preferably hydrogen, hydroxyl, amino, nitro, cyano, carboxyl, carbamoyl, thiocarbamoyl, fluorine, chlorine, bromine, or one of the following moieties

—R⁶, —Q—R⁶, —N(SO₂R⁶)₂, —CQ¹—R⁷, —CQ¹13 Q²—R⁶, —CQ¹—NH—R⁶, —Q²—CQ¹—R⁶, Q²—CQ¹—Q²—R⁶, —N(SO₂—R⁶)(CQ¹—R⁶),

where particularly preferably

Q is O, S, SO or SO₂,

Q¹ and Q² are independently O or S, and

R⁶ is methyl, ethyl, n- or i-propyl, each of which is optionally substituted by cyano, fluorine, chlorine, methoxy, ethoxy, methylthio, ethylthio, acetyl, propionyl, methoxycarbonyl, ethoxycarbonyl, methylaminocarbonyl or ethylaminocarbonyl,

or is propenyl, butenyl, propinyl or butinyl, each of which is optionally substituted by cyano, carboxyl, fluorine, chlorine, bromine, acetyl, propionyl, methoxycarbonyl, ethoxycarbonyl, n- or i- propoxycarbonyl, methylaminocarbonyl, ethylaminocarbonyl, n- or i-propylaminocarbonyl,

or is cyclopropyl, cyclobutyl, cyclopentyl, cyclohexyl, cyclopropylmethyl, cyclobutylmethyl, cyclopentylmethyl or cyclohexylmethyl, each of which is optionally substituted by cyano, carboxyl, fluorine, chlorine, methoxycarbonyl or ethoxycarbonyl,

or is phenyl, pyridin-2-yl, pyridin-3-yl, pyridin-4-yl, thien-2-yl, thien-3-yl or benzyl, each of which is optionally substituted by from one to three substituents selected from the group consisting of hydroxyl, mercapto, amino, cyano, carboxyl, carbamoyl, thiocarbamoyl, methyl, ethyl, trifluoromethyl, methoxy, ethoxy, difluoromethoxy, trifluoromethoxy, methylthio, ethylthio, difluoromethylthio, trifluoromethylthio, methylsulphinyl, ethylsulphinyl and methylsulphonyl.

R² is most preferably hydrogen.

R³ is most preferably fluorine.

R⁴ is most preferably cyano, bromine or trifluoromethyl.

If, for example, 1-(2-chloro-4-cyano-5-ethoxyphenyl)-3,6-dihydro-2,6-dioxo-4-difluoromethyl-1(2H)pyrimidine and 2-aminooxysulphonyl-1,3,5-trimethylbenzene are used as starting materials, the course of the reaction in the process of the invention can be outlined by the following scheme:

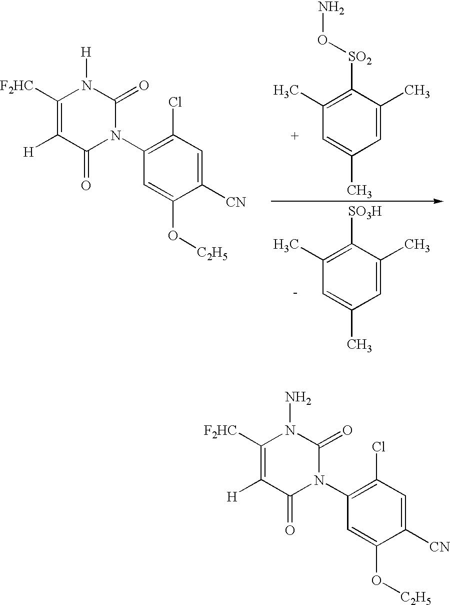

The 3-aryluracils to be used as starting materials in the process of the invention for the preparation of compounds of the general formula (I) are generally defined by the formula (II). In the general formula (II), R¹, R², R³, R⁴ and R⁵ preferably have those meanings which have already been given above in relation to the description of the compounds of the invention of the general formula (I) as preferable, more preferable, particularly preferable or most preferable for R¹, R², R³, R⁴ and R⁵. The starting materials of the general formula (II) are known and/or can be prepared by processes known per se (cf. EP-A-408382, EP-A-473551, EP-A-648749, U.S. Pat. No. 5169430, WO-A-91/00278, WO-A-95/29168, WO-A-95/30661, WO-A-96/35679).

The compound 2-aminooxysulphonyl-1,3,5-trimethylbenzene (O-mesitylene-sulphonylhydroxylamine) of the formula (III) to be used as a starting material in the process of the invention is also known and/or can be prepared by processes known per se (cf. J. Org. Chem. 1973 (38), 1239-1241; Synthesis 1972, 140; loc. cit. 1975, 788-789).

The process of the invention for preparing 1-amino-3-aryluracils is preferably carried out with the use of a reaction auxiliary. Useful reaction auxiliaries generally include the customary inorganic or organic bases or acid acceptors. These include, for example, acetates, amides, carbonates, hydrogencarbonates, hydrides, hydroxides or alkoxides of alkali metals or alkaline earth metals, such as sodium acetate, potassium acetate or calcium acetate, lithium amide, sodium amide, potassium amide or calcium amide, sodium carbonate, potassium carbonate or calcium carbonate, sodium hydrogencarbonate, potassium hydrogencarbonate or calcium hydrogencarbonate, lithium hydride, sodium hydride, potassium hydride or calcium hydride, lithium hydroxide, sodium hydroxide, potassium hydroxide or calcium hydroxide, sodium methoxide, ethoxide, n- or i-propoxide, n-, i-, s- or t-butoxide, or potassium methoxide, ethoxide, n- or i-propoxide, n-, i-, s- or t-butoxide; and also basic organic nitrogen compounds, for example trimethylamine, triethylamine, tripropylamine, tributylamine, ethyldiisopropylamine, N,N-dimethylcyclohexylamine, dicyclohexylamine, ethyldicyclohexylamine, N,N-dimethylaniline, N,N-dimethylbenzylamine, pyridine, 2-methyl-, 3-methyl-, 4-methyl-, 2,4-dimethyl-, 2,6-dimethyl-, 3,4-dimethyl- and 3,5-dimethylpyridine, 5-ethyl-2-methylpyridine, 4-dimethylaminopyridine, N-methylpiperidine, 1,4-diazabicyclo[2.2.2]octane (DABCO), 1,5-diazabicyclo[4.3.0]non-5-ene (DBN) or 1,8-diazabicyclo[5.4.0]undec-7-ene (DBU).

Preferred reaction auxiliaries include sodium carbonate and potassium carbonate, and also sodium hydrogencarbonate and potassium hydrogencarbonate.

The process of the invention for preparing the compounds of the general formula (I) is preferably carried out with the use of a diluent. Suitable diluents, as well as water, include above all inert organic solvents. These include in particular aliphatic, alicyclic or aromatic, optionally halogenated hydrocarbons, such as benzine, benzene, toluene, xylene, chlorobenzene, dichlorobenzene, petroleum ether, hexane, cyclohexane, dichloromethane, chloroform, carbon tetrachloride; ethers, such as diethyl ether, diisopropyl ether, methyl t-butyl ether, methyl t-pentyl ether, dioxane, tetrahydrofuran or ethylene glycol dimethyl or diethyl ether, ketones, such as acetone, butanone, methyl isobutyl ketone; nitriles, such as acetonitrile, propionitrile or butyronitrile; amides, such as N,N-dimethylformamide, N,N-dimethylacetamide, N-methylformanilide, N-methylpyrrolidone or hexamethylphosphoramide; esters such as methyl acetate or ethyl acetate, sulphoxides, such as dimethyl sulphoxide, alcohols, such as methanol, ethanol, n- or i-propanol, ethylene glycol monomethyl ether, ethylene glycol monoethyl ether, diethylene glycol monomethyl ether, diethylene glycol monoethyl ether, their mixtures with water or pure water.

More preferred diluents are aprotic polar organic solvents, for example dichloromethane, chloroform, diisopropyl ether, methyl t-butyl ether, methyl t-pentyl ether, dioxane, tetrahydrofuran, ethylene glycol dimethyl or diethyl ether, acetone, butanone, methyl isobutyl ketone, acetonitrile, propionitrile, butyronitrile, N,N-dimethylformamide, N,N-dimethylacetamide, N-methylformanilide, N-methylpyrrolidone, hexamethylphosphoramide, methyl acetate, ethyl acetate or dimethyl sulphoxide.

The reaction temperatures when operating the process of the invention can be varied within a wide range. In general, temperatures in the range from −50° C. to +80° C., preferably from −30° C. to +60° C., more preferably from −10° C. to +40° C., are employed.

The process of the invention is generally carried out under atmospheric pressure. However, it is also possible to carry out the process of the invention under increased or decreased pressure, in general from 0.1 bar to 10 bar.

To carry out the process of the invention, generally from 1 to 3 mol, preferably from 1.5 to 2.5 mol, of 2-aminooxy-1,3,5-trimethylbenzene (O-mesitylenesulphonylhydroxylamine) of the formula (III) are used per mole of 3-aryluracil of the general formula (II).

In a preferred embodiment, the 3-aryluracil of the general formula (II) and a reaction auxiliary in a suitable diluent are introduced as an initial charge and the 2-aminooxy-1,3,5-trimethylbenzene (O-mesitylenesulphonylhydroxylamine) of the formula (III) is added slowly. The addition can also take place in several portions spread over several hours. The reaction mixture is stirred until the end of the reaction.

The workup can be carried out by customary methods. For example, the reaction mixture is poured into approximately the same volume of 10% aqueous ammonium chloride solution and then extracted with an organic solvent which is virtually immiscible with water. The organic phase is then washed with water or with a saturated aqueous sodium chloride solution, dried and filtered. The solvent is carefully distilled off from the filtrate under reduced pressure. The crude product obtained as the residue can be further purified by customary methods.

The 1-amino-3-aryluracils to be prepared by the process of the invention can be used as active ingredients in agrochemicals or as intermediates for preparing active ingredients (cf. WO-A-94/04511, WO-A-95/29168, WO-A-96/36614, WO-A-97/05116, WO-A-98/06706, WO-A-98/25909).

The determination of the logP value given in Example 1 was carried out according to EEC Directive 79/831 Annex V.A8 by HPLC (High Performance Liquid Chromatography) using a reversed-phase column (C 18). Temperature: 43° C. (a) Eluents for determination in the acid range: 0.1% aqueous phosphoric acid, acetonitrile; linear gradient from 10% acetonitrile to 90% acetonitrile—corresponding measurements are marked in Table 1 by ^{a}).

(b) Eluents for determination in the neutral range: 0.01% molar aqueous phosphate buffer solution, acetonitrile; linear gradient of 10% acetonitrile to 90% acetonitrile—corresponding measurements are marked in Table 1 by ^{b}).

Calibration was carried out using unbranched alkan-2-ones (having from 3 to 16 carbon atoms), whose logP values are known (determination of logP values using the retention times by linear interpolation between two consecutive alkanones).

### PREPARATION EXAMPLES

### Example 1

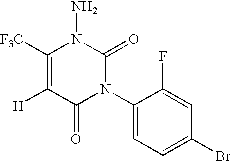

0.50 g (1.5 mmol) of 3-(4-bromo-2-fluorophenyl)-6-trifluoromethyl-2,4-(1H,3H)-pyrimidinedione, 0.24 g (1.8 mmol) of potassium carbonate and 50 ml of tetrahydrofuran are introduced as an initial charge, and 0.60 g (2.8 mmol) of 2-aminooxysulphonyl-1,3,5-trimethylbenzene (O-mesitylenesulphonylhydroxylamine) is added to the mixture at 0° C. with stirring. After removal of the coolant, the reaction mixture is then stirred for 18 hours at room temperature (about 20° C.). The reaction mixture is then poured into a 10% aqueous ammonium chloride solution and extracted with ethyl acetate in a separating funnel. The organic phase is separated off, washed with saturated aqueous sodium chloride solution, dried over sodium sulphate and filtered. The solvent is carefully distilled out of the filtrate under reduced pressure. The residue is digested with i-propanol and the crystalline product is isolated by filtration with suction.

0.27 g (49% of the theory) of 1-amino-3-(4-bromo-2-fluorophenyl)-6-trifluoromethyl-2,4-(1H,3H)-pyrimidinedione is obtained.

LogP=2.41 (at pH=2.3).

¹H NMR (DMSO-d₆, δ): 6.42 ppm (s).

### Example 2

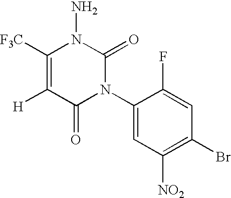

1.0 g (2.5 mmol) of 3-(4-bromo-2-fluoro-5-nitrophenyl)-6-trifluoromethyl-2,4-(1H,3H)-pyrimidinedione, 0.30 g (3.8 mmol) sodium hydrogencarbonate and 0.5 g sodium sulphate in 50 ml dichloromethane are introduced as an initial charge and this mixture is stirred for 15 minutes at room temperature (about 20° C.). 0.90 g (4.2 mmol) of 2-aminooxysulphonyl-1,3,5-trimethylbenzene (O-mesitylene-sulphonylhydroxylamine) is added with stirring and the reaction mixture is stirred for 18 hours at room temperature. 0.2 g (0.9 mmol) of 2-aminooxysulphonyl-1,3,5-trimethylbenzene is then added and the mixture is stirred for a further 2 hours at room temperature. A further 0.2 g (0.9 mmol) of 2-aminooxysulphonyl-1,3,5-tri-methylbenzene is then added and the mixture is stirred for a further 2 hours at room temperature. The mixture is then poured into an approximately equal volume of 1 N hydrochloric acid. Extraction is then carried out three times using ethyl acetate; the united organic phases are dried over sodium sulphate and filtered. The filtrate is concentrated in a water pump vacuum and the residue (1.4 g) is purified by column chromatography (silica gel, chloroform/ethyl acetate, 2:1 v:v). After distilling off the eluents in a water pump vacuum, the residue (1.0 g) is digested with diethyl ether/diisopropyl ether and the crystalline product is isolated by filtration with suction.

0.65 g (63% of the theory) of 1-amino-3-(4-bromo-2-fluoro-5-nitrophenyl)-6-tri-fluoromethyl-2,4-(1H,3H)-pyrimidinedione of melting point 165° C. is obtained.

¹H NMR (DMSO-d₆, δ): 6.47 ppm (s).

### Example 3

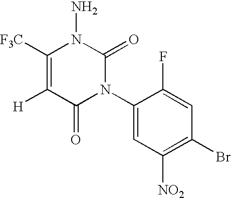

1.0 g (2.5 mmol) of 3-(4-bromo-2-fluoro-5-nitrophenyl)-6-trifluoromethyl-2,4-(1H,3H)-pyrimidinedione, 0.30 g (3.8 mmol) sodium hydrogencarbonate and 1.0 g of sodium sulphate in 50 ml ethyl acetate are introduced as an initial charge and 0.90 g (4.2 mmol) of 2-aminooxysulphonyl-1,3,5-trimethylbenzene (O-mesitylene-sulphonylhydroxylamine) is added to the mixture with stirring after stirring for 15 minutes at room temperature (about 20° C.). The reaction mixture is stirred for 18 hours at room temperature. 0.2 g (0.9 mmol) of 2-aminooxysulphonyl-1,3,5-trimethylbenzene is then added and the mixture is stirred for a further 2 hours at room temperature. A further 0.2 g (0.9 mmol) of 2-aminooxysulphonyl-1,3,5-trimethylbenzene is then added and the mixture is stirred for a further 2 hours at room temperature. The mixture is then poured into an approximately equal volume of 1 N hydrochloric acid. Extraction is then carried out three times using ethyl acetate; the united organic phases are dried over sodium sulphate and filtered. The filtrate is concentrated in a water pump vacuum, the residue (1.3 g) digested with diethyl ether and the crystalline product isolated by filtration with suction.

0.80 g (77.5% of the theory) of 1-amino-3-(4-bromo-2-fluoro-5-nitrophenyl)-6-trifluoromethyl-2,4-(1H,3H)-pyrimidinedione of melting point 165° C. is obtained.

¹H NMR (DMSO-d₆, δ): 6.47 ppm (s).

### Examples of Subsequent Reactions/preparation of Further Intermediates or Active Ingredients

### Example 4

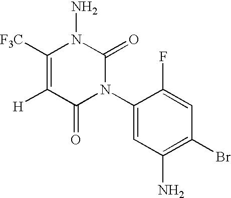

1.0 g (2.4 mmol) of 1-amino-3-(4-bromo-2-fluoro-5-nitrophenyl)-6-trifluoromethyl-2,4-(1H,3H)-pyrimidinedione in 20 ml acetic acid (containing 10% water) are introduced as an initial charge and admixed with 0.8 g (14 mmol) of iron divided into 6 portions at 50° C. The reaction mixture is stirred for 3 hours at 50° C. and then filtered with suction through kieselguhr/sand. The filtrate is shaken with ethyl acetate/water, the organic phase washed with water and filtered with suction through silica gel. The filtrate is concentrated in a water pump vacuum, the residue digested with diisopropyl ether and the crystalline product isolated by filtration with suction.

0.57 g (62% of the theory) of 1-amino-3-(5-amino-4-bromo-2-fluoro-5-phenyl)-6-tri-fluoromethyl-2,4-(1H,3 H)-pyrimidinedione is obtained.

¹H NMR (DMSO-d₆, δ): 6.36 ppm (s).

### Example 5

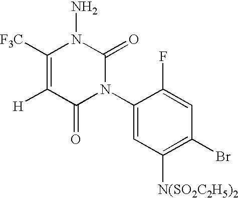

0.50 g (1.3 mmol) of 1-amino-3-(5-amino-4-bromo-2-fluoro-5-phenyl)-6-trifluoromethyl-2,4-(1H,3H)-pyrimidinedione, 0.3 g (3 mmol) of triethylamine and 20 ml of dichloromethane are introduced as an initial charge and admixed dropwise with a solution of 0.25 g (2 mmol) of ethanesulphonyl chloride in 5 ml of dichloromethane at 0° C. with stirring. The reaction mixture is stirred for 4 hours at 0° C. After adding a further 0.45 g of ethanesulphonyl chloride, the reaction mixture is stirred for a further 18 hours at room temperature (about 20° C.). It is then shaken with 1 N aqueous hydrochloric acid/dichloromethane, the organic phase is washed with 1 N hydrochloric acid, dried over sodium sulphate and filtered. The filtrate is concentrated in a water pump vacuum, the residue digested with diisopropyl ether and the crystalline product isolated by filtration with suction.

0.56 g (76% of the theory) of 1-amino-3-[5-(bis-ethylsulphonylamino)-4-bromo-2-fluoro-5-phenyl]-6-trifluoromethyl-2,4-( 1 H,3H)-pyrimidinedione is obtained.

¹H NMR (DMSO-d₆, δ): 6.44 ppm (s).

### Example 6

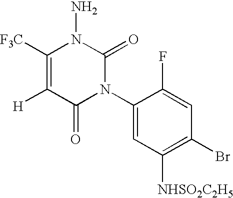

A mixture of 0.50 g (0.88 mmol) of 1-amino-3-[5-(bis-ethylsulphonylamino)-4-bromo-2-fluoro-5-phenyl]-6-trifluoromethyl-2,4-(1H,3H)-pyrimidinedione, 0.15 g (1 mmol) of sodium hydrogencarbonate, 50 ml of water and 50 ml of acetone is stirred for 18 hours at room temperature (about 20° C.) and then for 4 hours at 60° C. The acetone is then substantially distilled off in a water pump vacuum, the residue diluted with water to about twice its initial volume and set to pH=1 by addition of aqueous 1 N hydrochloric acid. It is then shaken with ethyl acetate, the organic phase dried over sodium sulphate and filtered. The solvent is carefully distilled off from the filtrate under reduced pressure.

0.25 g (60% of the theory) of 1-amino-3-[5-(ethylsulphonylamino)-4-bromo-2-fluoro-5-phenyl]-6-trifluoromethyl-2,4-(1H,3H)-pyrimidinedione is obtained.

¹H NMR (DMSO-d₆, δ): 6.41 ppm (s).

### Example 7

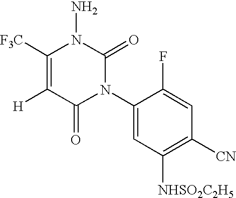

1.90 g (4.0 mmol) of 1-amino-3-[5-(ethylsulphonylamino)-4-bromo-2-fluoro-5-phenyl]-6-trifluoromethyl-2,4-(1H,3H)-pyrimidinedione and 0.43 g (4.8 mmol) of copper(I) cyanide in 10 ml N-methylpyrrolidone are introduced as an initial charge. In order to remove remaining water, 5 ml of N-methylpyrrolidone are distilled off at from 85 to 90° C. (at from 2 mbar to 4 mbar). The reaction mixture is then heated for 270 minutes at from 160° C. to 165° C. A further 4 ml of N-methylpyrrolidone are distilled off under reduced pressure. After cooling to from 10 to 15° C., 15 ml of ethyl acetate and also 1.0 g (6.2 mmol) of iron(III) chloride in 5 ml water and 0.5 ml of conc. hydrochloric acid are added and the mixture is stirred for 30 minutes at room temperature (about 20° C.). The organic phase is separated off, the aqueous phase subjected to further extraction using ethyl acetate, the united organic phases are washed with water, dried over sodium sulphate and filtered. The solvent is distilled off from the filtrate under reduced pressure.

1.15 g of crude product are obtained, which according to HPLC (High Performance Liquid Chromatography) contains 87.3% of 1-amino-3-[4-cyano-5-(ethylsulphonyl-amino)-2-fluoro-5-phenyl]-6-trifluoromethyl-2,4-(1H,3H)-pyrimidinedione (60% of the theory).

### Example 8

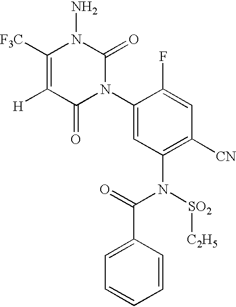

0.50 g (1 mmol) of N-benzoyl-N-[2-cyano-5-(2,6-dioxo-4-trifluoromethyl-3,6-dihydro-1-(2H)-pyrimidinyl)-4-fluorophenyl]ethanesulphonamide, 0.12 g (1 mmol) of sodium hydrogencarbonate and 0.5 g of sodium sulphate in 25 ml of methylene chloride are introduced as an initial charge and after stirring for 15 minutes at room temperature (about 20° C.), 0.12 g (0.5 mmol) of 2-aminooxysulphonyl-1,3,5-trimethylbenzene (O-mesitylenesulphonylhydroxylamine) is added to this mixture with stirring. The reaction mixture is stirred for 30 minutes at room temperature. A further 0.12 g of 2-aminooxysulphonyl-1,3,5-trimethylbenzene is then added and the mixture is stirred for a further 30 minutes. The addition of 2-aminooxysulphonyl-1,3,5-trimethylbenzene and stirring for 30 minutes are repeated twice more. The mixture is then stirred for a further 15 hours at room temperature. It is then added to an approximately equal volume of 1 N hydrochloric acid and extracted twice with ethyl acetate. The organic extraction solutions are united, dried over sodium sulphate and filtered. The filtrate is concentrated under reduced pressure, the residue digested with diethyl ether/petroleum ether and the crystalline product isolated by suction filtration.

0.40 g (78% of the theory) of N-benzoyl-N-[2-cyano-5-(3-amino-2,6-dioxo-4-trifluoromethyl-3,6-dihydro- 1-(2H)-pyrimidinyl)-4-fluorophenyl]ethanesulphonamide is obtained.

LogP=2.82 (at pH=2.3).

1. Process for preparing 1-amino-3-aryluracils of the formula (I) 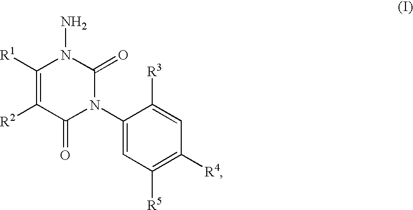
in which
R¹ is optionally substituted alkyl,
R² is hydrogen, nitro, cyano, halogen or optionally substituted alkyl,
R³ is hydrogen, nitro, cyano or halogen,
R⁴ is hydrogen, nitro, cyano, carbamoyl, thiocarbamoyl, hydroxyl or halogen, or optionally substituted alkyl, alkoxy or benzoyloxy,
R⁵ is hydrogen, hydroxyl, mercapto, amino, hydroxyamino, nitro, cyano, carboxyl, carbamoyl, thiocarbamoyl or halogen, or is one of the following moieties
—R⁶, —Q—R⁶, —NH—R⁶, —NH—O—R⁶, —NH—SO₂—R⁶, —N(SO₂R⁶)₂, —CQ¹—R⁷, —CQ¹—Q²—R⁶, —CQ¹—NH—R⁶, —Q²—CQ¹—R⁶, —Q²—CQ¹—Q²—R⁶, —NH—CQ¹—R⁶, —N(SO₂—R⁶)—(CQ¹—R⁶), —NH—CQ¹—Q²—R⁶, —Q²—CQ¹—NH—R⁶
where
Q is O, S, SO or SO₂,
Q¹ and Q² are independently O or S, and
R⁶ is alkyl alkenyl, alkinyl, cycloalkyl, cycloalkylalkyl, aryl, arylalkyl, heterocyclyl or heterocyclylalkyl, each of which is optionally substituted,
which comprises reacting 3-aryluracils of the formula (II) 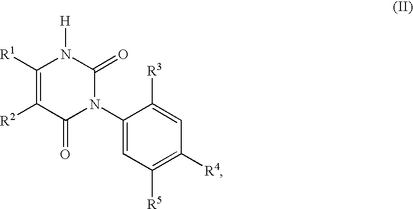
in which
R¹, R², R³, R⁴ and R⁵ have the above meanings, with 2-aminooxysulphonyl-1,3,5-trimethylbenzene (O-mesitylenesulphonyl-hydroxylamine) of the formula (III) 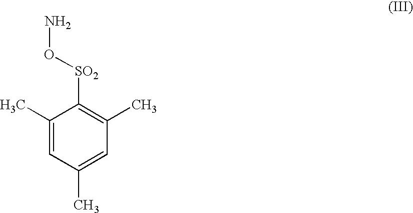
optionally in the presence of a reaction auxiliary and optionally in the presence of a diluent at temperatures in the range from −50° C. to 80° C.

2. Process according to claim 1, characterized in that compounds of the formula (II) are used as starting materials, in which
R¹ is optionally halogen-substituted alkyl having from 1 to 4 carbon atoms,
R² is hydrogen, nitro, cyano, halogen or optionally halogen-substituted alkyl having from 1 to 4 carbon atoms,
R³ is hydrogen, nitro, cyano, fluorine, chlorine or bromine,
R⁴ is hydrogen, nitro, cyano, carbamoyl, thiocarbamoyl, hydroxyl or halogen, or is alkyl or alkoxy having from 1 to 4 carbon atoms, each of which is optionally substituted by halogen, or is optionally halogen-, C₁-C₄-alkyl- or C₁-C₄-alkoxy-substituted benzoyloxy and
R⁵ is hydrogen, hydroxyl, mercapto, amino, hydroxyamino, nitro, cyano, carboxyl, carbamoyl, thiocarbamoyl, halogen, or is one of the following moieties
—R⁶, —Q—R⁶, —NH—R⁶, —NH—O—R⁶, —NH—SO₂—R⁶, —N(SO₂R⁶)₂, —CQ¹—R⁷,
—CQ¹—Q²—R⁶, —CQ¹—NH—R⁶, —Q²—CQ¹—R⁶, —Q²—CQ¹—Q²—R⁶,
—NH—CQ¹—R⁶, —N(SO₂—R⁶)(CQ¹—R⁶), —NH—CQ¹—Q²—R⁶,
—Q²—CQ —NH—R⁶,
where
Q is O, S, SO or SO₂,
Q¹ and Q² are independently O or S, and
R⁶ is alkyl having from 1 to 6 carbon atoms, which is optionally substituted by cyano, halogen, C₁-C₄-alkoxy, C₁-C₄-alkylthio, C₁-C₄-alkylcarbonyl, C₁-C₄-alkoxycarbonyl or C₁-C₄-alkylaminocarbonyl,
or is alkenyl or alkinyl having from 2 to 6 carbon atoms, each of which is optionally substituted by cyano, carboxyl, halogen, C₁-C₄-alkylcarbonyl, C₁-C₄-alkoxycarbonyl or C₁-C₄-alkyl-aminocarbonyl,
or is cycloalkyl or cycloalkylalkyl having from 3 to 6 carbon atoms in the cycloalkyl group and optionally from 1 to 4 carbon atoms in the alkyl part, each of which is optionally substituted by cyano, carboxyl, halogen, C₁-C₄-alkylcarbonyl or C₁-C₄-alkoxycarbonyl,
or is aryl or arylalkyl having 6 or 10 carbon atoms in the aryl group and optionally from 1 to 4 carbon atoms in the alkyl part, each of which is optionally substituted by from one to three substituents selected from the group consisting of hydroxyl, mercapto, amino, cyano, carboxyl, carbamoyl, thiocarbamoyl, C₁-C₄-alkyl, C₁-C₄-halogenalkyl, C₁-C₄-alkoxy, C₁-C₄-halogenalkoxy, C₁-C₄-alkylthio, C₁-C₄-halogenalkylthio, C₁-C₄-alkylsulphinyl, C₁-C₄-alkylsulphonyl, C₁-C₄-alkylamino and dimethylamino,
or is heterocyclyl or heterocyclylalkyl having from 2 to 6 carbon atoms and from 1 to 3 nitrogen atoms and/or 1 or 2 oxygen atoms and/or a sulphur atom in the heterocyclyl group and optionally from 1 to 4 carbon atoms in the alkyl part, each of which is optionally substituted by from one to three substituents selected from the group consisting of hydroxyl, mercapto, amino, cyano, carboxyl, carbamoyl, thiocarbamoyl, C₁-C₄-alkyl, C₁-C₄-halogenalkyl, C₁-C₄-alkoxy, C₁-C₄-halogenalkoxy, C₁-C₄-alkylthio, C₁-C₄-halogenalkylthio, C₁-C₄-alkylsuphinyl, C₁-C₄-alkylsulphonyl, C₁-C₄-alkylamino and dimethylamino.

3. Process according to claim 1, characterized in that compounds of the formula (II) are used as starting materials in which
R¹ is methyl, ethyl, n- or i-propyl, each of which is optionally substituted by fluorine and/or chlorine,
R² is hydrogen, nitro, cyano, fluorine, chlorine or bromine, or is methyl or ethyl, each of which is optionally substituted by fluorine and/or chlorine,
R³ is hydrogen, fluorine or chlorine,
R⁴ is hydrogen, nitro, cyano, carbamoyl, thiocarbamoyl, hydroxyl, fluorine, chlorine or bromine, or is methyl or methoxy, each of which is optionally substituted by fluorine and/or chlorine, and
R⁵ is hydrogen, hydroxyl, mercapto, amino, hydroxyamino, nitro, cyano, carboxyl, carbamoyl, thiocarbamoyl, fluorine, chlorine, bromine, iodine, or one of the following moieties
—R⁶, —Q—R⁶, —NH—R⁶, —NH—O—R⁶, —NH—SO₂—R⁶, —N(SO₂R⁶)₂, —CQ¹—R⁷, —CQ¹—Q²—R⁶, —CQ¹—NH—R⁶, —Q²—CQ¹—R⁶, —Q²CQ¹—Q²—R⁶, —NH—CQ¹—R⁶, —N(SO₂—R⁶)(CQ¹—R⁶), —NH—CQ¹—Q²—R⁶, —Q²—CQ¹—NH—R⁶,
where
Q is O, S, SO or SO₂,
Q¹ and Q² are independently O or S, and
R⁶ is methyl, ethyl, n- or i-propyl, n-, i-, s- or t-butyl, each of which is optionally substituted by cyano, fluorine, chlorine, methoxy, ethoxy, methylthio, ethylthio, acetyl, propionyl, methoxycarbonyl, ethoxycarbonyl, methylaminocarbonyl or ethylaminocarbonyl,
or is propenyl, butenyl, propinyl or butinyl, each of which is optionally substituted by cyano, carboxyl, fluorine, chlorine, bromine, acetyl, propionyl, n- or i-butyroyl, methoxycarbonyl, ethoxycarbonyl, n- or i-propoxycarbonyl, methylaminocarbonyl, ethylaminocarbonyl or n- or i-propylaminocarbonyl,
or is cyclopropyl, cyclobutyl, cyclopentyl, cyclohexyl, cyclopropylmethyl, cyclobutylmethyl, cyclopentylmethyl or cyclohexylmethyl, each of which is optionally substituted by cyano, carboxyl, fluorine, chlorine, bromine, acetyl, propionyl, methoxycarbonyl or ethoxycarbonyl,
or is phenyl, benzyl or phenylethyl, each of which is optionally substituted by from one to three substituents selected from the group consisting of hydroxyl, mercapto, amino, cyano, carboxyl, carbamoyl, thiocarbamoyl, methyl, ethyl, trifluoromethyl, methoxy, ethoxy, difluoromethoxy, trifluoromethoxy, methylthio, ethylthio, difluoromethylthio, trifluoromethylthio, methylsulphinyl, ethylsulphinyl, methylsulphonyl, methylamino, ethylamino and dimethylamino,
or is heterocyclyl or heterocyclylalkyl selected from the group consisting of oxiranyl, oxetanyl, furyl, tetrahydrofuryl, dioxolanyl, thienyl, tetrahydrothienyl, pyrrolyl, pyrazolyl, imidazolyl, triazolyl, oxazolyl, isoxazolyl, thiazolyl, isothiazolyl, oxadiazolyl, thiadiazolyl, pyridinyl, pyrimidinyl, triazinyl, pyrazolylmethyl, furylmethyl, thienylmethyl, oxazolylmethyl, isoxazolylmethyl, thiazolylmethyl, pyridinylmethyl and pyrimidinylmethyl, each of which is optionally substituted by one or two substituents selected from the group consisting of hydroxyl, mercapto, amino, cyano, carboxyl, carbamoyl, thiocarbamoyl, methyl, ethyl, n- or i-propyl, n-, i-, s- or t-butyl, difluoromethyl, dichloromethyl, trifluoromethyl, trichloromethyl, chlorodifluoromethyl, fluorodichloromethyl, methoxy, ethoxy, difluoromethoxy, trifluoromethoxy, methylthio, ethylthio, difluoromethylthio, trifluoromethylthio, methylsulphinyl, ethylsulfphinyl, methylsulphonyl, ethylsulphonyl, methylamino, ethylamino and dimethylamino.

4. Process according to claim 1, characterized in that compounds of the formula (II) are used as starting materials in which
R¹ is trifluoromethyl,
R² is hydrogen, chlorine or methyl,
R³ is fluorine or chlorine,
R⁴ is cyano, carbamoyl, thiocarbamoyl, hydroxyl, fluorine, chlorine, bromine or trifluoromethyl and
R⁵ is hydrogen, hydroxyl, amino, nitro, cyano, carboxyl, carbamoyl, thiocarbamoyl, fluorine, chlorine, bromine, or is one of the following moieties
—R⁶, —Q—R⁶, —N(SO₂R⁶)₂, —CQ¹—R⁷, —CQ¹—Q²—R⁶, —CQ¹—NH—R⁶, —Q²—CQ¹—R⁶, —Q²—CQ¹—Q²—R⁶, —N(SO₂—R⁶)(CQ¹—R⁶),
where
Q is O, S, SO or SO₂,
Q¹ and Q² are independently O or S, and
R⁶is methyl, ethyl, n- or i-propyl, each of which is optionally substituted by cyano, fluorine, chlorine, methoxy, ethoxy, methylthio, ethylthio, acetyl, propionyl, methoxycarbonyl, ethoxycarbonyl, methylaminocarbonyl or ethylaminocarbonyl,
or is propenyl, butenyl, propinyl or butinyl, each of which is optionally substituted by cyano, carboxyl, fluorine, chlorine, bromine, acetyl, propionyl, methoxycarbonyl, ethoxycarbonyl, n- or i- propoxycarbonyl, methylaminocarbonyl, ethylaminocarbonyl, n- or i-propylaminocarbonyl,
or is cyclopropyl, cyclobutyl, cyclopentyl, cyclohexyl, cyclopropylmethyl, cyclobutylmethyl, cyclopentylmethyl or cyclohexylmethyl, each of which is optionally substituted by cyano, carboxyl, fluorine, chlorine, methoxycarbonyl or ethoxycarbonyl,
or is phenyl, pyridin-2-yl, pyridin-3-yl, pyridin-4-yl, thien-2-yl, thien-3-yl or benzyl, each of which is optionally substituted by from one to three substituents selected from the group consisting of hydroxyl, mercapto, amino, cyano, carboxyl, carbamoyl, thiocarbamoyl, methyl, ethyl, trifluoromethyl, methoxy, ethoxy, difluoromethoxy, trifluoromethoxy, methylthio, ethylthio, difluoromethylthio, trifluoromethylthio, methylsulphinyl, ethylsulphinyl and methylsulphonyl.

5. Process according to any of claims 1 to 4, characterized in that compounds of the formula (II) are used as starting materials in which
R² is hydrogen,
R³ is fluorine and
R⁴ is cyano, bromine or trifluoromethyl.
